(19) United States
(12) Patent Application Publication
(10) Pub. No.: US 2026/0293932 A1
(43) Pub. Date: Oct. 1, 2026

(54) FERMENTED FOODS AND METHODS FOR MAKING SAME

(71) Applicant: MATR FOODS APS, Nordhavn (DK)

(72) Inventors: Morten Otto Alexander SOMMER, Copenhagen (DK); Nikolaj Fackler MADSEN, Copenhagen (DK); Cristina MEGIAS, Copenhagen (DK)

(21) Appl. No.: 18/880,440

(22) PCT Filed: Jul. 13, 2023
(86) PCT No.: PCT/EP2023/069428

§ 371 Date: Dec. 31, 2024

(30) Foreign Application Priority Data
Jul. 14, 2022 (EP) 22184925.0

(51) Int. Cl. A23J 3/22; A23J 3/14; A23J 3/18; A23L 7/104; A23L 11/00; A23L 11/50; A23L 19/12

(52) U.S. Cl. CPC A23J 3/227; A23J 3/14; A23J 3/18; A23L 7/104; A23L 11/05; A23L 11/50; A23L 19/13

(57) ABSTRACT

The present disclosure relates to a method for preparing a fermented edible product enriched or improved in one or more properties selected from flavour, aroma, texture, cooking experience, visual appearance, nutritional and/or dietary value, spoilage time, and/or carbon footprint comprising:
a) Providing one or more edible ingredients.
b) subjecting the one or more edible ingredients to one or more preparation steps to form a fermentation substrate.
c) contacting the fermentation substrate with one or more microorganisms under conditions allowing the microorganisms to colonize and proliferate on/in the fermentation substrate; and
d) isolating the fermentation substrate and the one or more microorganisms into the edible product.

### TECHNICAL FIELD

The present application concerns fermented edible products which are improved in one or more quality properties of flavour, aroma, texture, cooking experience, visual appearance, nutritional and/or dietary value, spoilage time, and/or carbon foot-print, as well as methods for producing such fermented edible products.

### BACKGROUND

The practice of food fermentation and its diverse techniques can be traced back to ancient times and is currently prevalent across various food cultures worldwide. One must only consider the examples of beer, cheese, and yoghurt to get an idea about the magnitude of the use fermentation in the food and beverage industry. However, there is now an increased demand for foods based on vegetable sources. Increasingly, consumers prioritize food options that align with climate and ecological sustainability, specifically emphasizing the utilization of raw materials that would otherwise be classified as waste streams or currently unfit for human consumption or food production. There exists a demand to create high-quality food options based on plant sources, while simultaneously delivering the desired textures, flavours, nutrients, and other characteristics reminiscent of meat. Additionally, it is preferable to utilize vegetable raw materials sourced from agricultural or industrial waste streams (upcycling) either partially or entirely. The existing technologies used for meat alternatives are inadequate or unappealing due to their reliance on artificial additives and isolated proteins to achieve meat-like textures and flavours.

### SUMMARY

The methods and edible products described herein provide improvements that address the drawbacks and limitations of existing approaches. By fermenting vegetable raw materials, these methods enable the creation of meat-like properties while simultaneously delivering superior health and environmental benefits.

Accordingly, in a first aspect the present disclosure provides a method for preparing a fermented edible product, optionally enriched or improved in one or more properties selected from flavour, aroma, texture, cooking experience, visual appearance, nutritional and/or dietary value, spoilage time, and/or carbon footprint, comprising:
a) providing one or more edible ingredients;
b) subjecting the one or more edible ingredients to one or more preparation steps to form a fermentation substrate;
c) contacting the fermentation substrate with one or more microorganism under conditions allowing the microorganism to colonize and proliferate on/in the fermentation substrate; and
d) isolating the fermentation substrate and the one or more microorganisms into the edible product.

In a further aspect described herein is a fermented edible product comprising one or more filamentous fungi or their mycelium and 10% wt to 30% wt, optionally 15% wt to 25% wt, optionally 19% wt to 21% wt of oats, potato, red beet, P. sativum peas, and L. albus lupin seeds.

### DESCRIPTION OF DRAWINGS AND FIGURES

The FIGURES included herein are illustrative and simplified for clarity. They merely show details that are essential to the understanding of the invention, while other details may have been left out.

The FIGURES and drawings included herein depict the content described in this document.

FIG. 1 shows a flow chart for the preparation and mixtures of ingredients as described herein.

### INCORPORATION BY REFERENCE

All publications, specifically, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

The features and advantages of the present invention are readily apparent to a person skilled in the art by the below detailed description of embodiments and examples of the invention with reference to the figures and drawings included herein.

### Definitions

Percentages given herein as “% wt” refers to weight percent. Weight percents eg. for edible ingredients are in some embodiments calculated as the percentage of a substance in the natural form of the ingredient, such as raw unpeeled beets, while weight percents in other embodiments are calculated as the percentage of a substance in an ingredient which is processed such as by peeling, shredding, drying etc.

### Method

The first aspect provided for herein concerns a method for preparing a fermented edible product (food or feed), optionally enriched or improved in one or more properties selected from: flavour, aroma, texture, cooking experience, visual appearance, nutritional and/or value, spoilage time, and/or carbon footprint comprising:
a) Providing one or more edible ingredients;
b) subjecting the one or more edible ingredients to one or more preparation steps to form a fermentation substrate;
c) contacting the fermentation substrate with one or more microorganism under conditions allowing the microorganism to colonize and proliferate on/in the fermentation substrate; and
d) isolating the fermentation substrate and the one or more microorganisms into the edible product.

In another aspect provided for herein concerns a method for preparing a fermented edible product (food or feed) comprising:
a) Providing one or more edible ingredients;
b) subjecting the one or more edible ingredients to one or more preparation steps to form a fermentation substrate;
c) contacting the fermentation substrate with one or more microorganism under conditions allowing the microorganism to colonize and proliferate on/in the fermentation substrate; and
d) isolating the fermentation substrate and the one or more microorganisms into the edible product.

The edible product is typically a food, a pet food, or an animal feed composition, particularly a food composition, since for foods for human consumption there are particularly high requirements for flavour, aroma, texture, cooking experience, visual appearance, nutritional and/or dietary value, spoilage time, and/or carbon footprint.

Although the one or more edible ingredients can be sourced from any suitable source, the method is particularly useful when sourcing edible ingredients from industrial or agricultural waste streams which are not usually used or even suitable for human consumption due to lack of appropriate properties. One example of such edible ingredients is Brewers Spent Grain (BSG, typically barley or wheat), which is the cereal residue remaining after the cereal has been used for brewing of for example beer or other beverages. BSG contains, among other things, leftover starch, sugars, and fibres not spent during the brewing process. However, BSG is not very attractive as a food ingredient for humans. Therefore, prior to the availability of the method provided for herein, BSG has typically been used for animal fodder or incinerated for heat.

The choices of edible ingredients and the microbial fermentation, whether separately or together, all influence the properties of the edible product in particular flavour, aroma, texture, cooking experience, visual appearance, nutritional and/or dietary value, spoilage time, and/or carbon footprint. Accordingly in further embodiments, the edible ingredients can comprise one or more of:
a) a dry component comprising i) starch providing nutrient to the one or more microorganism and
   ii) fibres providing nutritional and/or dietary value to the edible product, the dry component being capable of absorbing moisture from other edible ingredients;
b) a high-moisture and high starch component providing for the formation of gelatinized starch, binding other edible ingredients together and providing nutrient to the one or more microorganisms, as well as texture to the edible product;
c) a component providing minerals and vitamins for the one or more microorganisms as well as a natural colour to the edible product;
d) a high-protein and low moisture component providing flavour and firmness and structure to the texture of the edible product; or
e) a high starch component providing sustained nutrients for the one or more microorganism and providing softness to the texture of the edible product.

The choice ingredients can be based on the ingredient's individual nutritional components, and their combined qualities as a growth medium for the fungal culture used.

In some embodiments, the one or more edible ingredients comprise at least 2, such as at least 3, such as at least 4, such as at least 5 components selected from the dry component, the high-moisture and high-starch component, the component providing minerals and vitamins, the high-protein and low-moisture component, and/or the high-starch component. In a special embodiment, the one or more edible ingredients comprise all 5 ingredients selected from the dry component, the high-moisture and high-starch component, the component providing minerals and vitamins, the high-protein and low-moisture component, or the high-starch component.

In more specific embodiments, the edible ingredients are selected from cereal grains, root vegetables, and legumes or a combination or extract thereof have proved to be particularly useful.

The cereal grain is very useful as the dry component, in particular cereals selected from maize (corn), rice, wheat, barley, sorghum, millet, oats, triticale, rye, and/or fonio. In some embodiments the cereal grain is spent grain from the fermentation of alcoholic beverages, brewers spent grain (BSG). More specifically the use of oats as cereal grain is particularly attractive. The one or more edible ingredients preferably comprise 5% wt to 60% wt of cereal grain, such as 5% wt to 10% wt, such as 10% wt to 15% wt, such as 15% wt to 20% wt such as 20% wt to 25% wt, such as 25% wt to 30% wt, such as 30% wt to 35% wt, such as 35% wt to 40% wt, such as 45% wt to 50% wt, such as 55% wt to 60% wt, optionally 19% wt to 21% wt of cereal grain, particularly oats.

The root vegetable is very useful as the high-moisture and high starch component and/or the component providing minerals and vitamins. The root vegetable can in particular be a modified plant stem vegetable, a root-like stem vegetable, or a true root vegetable.

Modified plant stem vegetables include Corms (bulbo-tuber/bulbotuber), Rhizomes or Tubers. Corm is suitably selected from Amorphophallus konjac (konjac), Colocasia esculenta (taro), Eleocharis dulcis (Chinese water chestnut), Ensete spp. (enset), Nymphaea spp. (waterlily), Pteridium esculentum, Sagittaria spp. (arrowhead or wapatoo), Typha spp., Xanthosoma spp. (malanga, cocoyam, tannia, yautia and other names), and/or Colocasia antiquorum (eddoe or Japanese potato). A rhizome is suitably selected from Curcuma longa (turmeric), Panax ginseng (ginseng), Arthropodium spp. (rengarenga, vanilla lily, and others), Canna spp. (canna), Cordyline fruticosa (ti), Maranta arundinacea (arrowroot), Nelumbo nucifera (lotus root), Typha spp. (cattail or bulrush), and/or Zingiber officinale (ginger, galangal). Tuber is suitably selected from Apios americana (hog potato or groundnut), Cyperus esculentus (tigernut or chufa), Dioscorea spp. (yams, ube), Dioscorea polystachya (Chinese yam or white ñame), Helianthus tuberosus (Jerusalem artichoke or sunchoke), Hemerocallis spp. (daylily), Lathyrus tuberosus (earthnut pea), Oxalis tuberosa (oca or New Zealand yam), Plectranthus edulis and P. esculentus (kembili, dazo, and others), Solanum tuberosum (potato), Stachys affinis (Chinese artichoke or crosne), Tropaeolum tuberosum (mashua or añu), and/or Ullucus tuberosus (ulluku). In a preferred embodiment the Tuber is of the genus Solanum, in particular Tuber of the species Solanum tuberosum (potato), such as the Solanum tuberosum red skin potato.

The root-like stem vegetable is suitably Zamia integrifolia (Florida arrowroot).

The true root vegetable is in some embodiments selected from taproot or tuberous root. The tuberous root vegetable can be selected from Amorphophallus galbra (yellow lily yam), Conopodium majus (pignut or earthnut), Dioscorea polystachya (nagaimo, Chinese yam, Korean yam, mountain yam), Hornstedtia scottiana (native ginger), Ipomoea batatas (sweet potato), Ipomoea costata (desert yam), Manihot esculenta (cassava or yuca or manioc), Mirabilis expansa (mauka or chago), Psoralea esculenta (breadroot, tipsin, or prairie turnip), and/or Smallanthus sonchifolius (yacón). The taproot vegetable can be selected from Arracacia xanthorrhiza (arracacha), Beta vulgaris (beet and mangelwurzel), Brassica spp. (kohlrabi, rutabaga, and turnip), Bunium persicum (black cumin), Burdock (Arctium, family Asteraceae), Carrot (Daucus carota subsp. sativus), Celeriac (Apium graveolens rapaceum), Daikon—the large East Asian white radish (Raphanus sativus var. longipinnatus), Dandelion (Taraxacum) spp., and/or Lepidium meyenii (maca). Preferably the taproot vegetable is Beta vulgaris, optionally subspecies vulgaris, in particular B. vulgaris var. conditiva (red beet).

In some embodiments of the described method the one or more edible ingredients comprise 5% wt to 60% wt root vegetable, such as 5% wt to 10% wt, such as 1 0% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt, 20% wt to 60% wt of a root vegetable, optionally 38% wt to 42% wt. In some embodiments, the one or more edible ingredients comprise 10% wt to 30% wt, optionally 15% wt to 25% wt, optionally 19% wt to 21% wt of Solanum tuberosum. In additional or alternative embodiments, the one or more edible ingredients can comprise optionally 15% wt to 2 5% wt, optionally 19% wt to 21% wt of B. vulgaris var. conditiva. In still further embodiments the one or more edible ingredients comprise 10% wt to 30% wt, optionally 15% wt to 25% wt, optionally 19% wt to 21% wt of Solanum tuberosum, and 10% wt to 30% wt, optionally 15% wt to 25% wt, optionally 19% wt to 21% wt of B. vulgaris var. conditiva.

The legume is very useful as the high-protein and low moisture component and/or the high starch component providing sustained nutrients for the one or more microorganisms, providing softness to the texture of the edible product. Useful legumes include and can be selected from the genera of Phaseolus, Pisum, Vigna, Cajanus, Lens, Cicer, Vicia, Arachis, Glycine, Macrotyloma, Mucuna, Lupinus, Ceratonia, Canavalia, Cyamopsis, Lablab, Psophocarpus, Clitoria, Lathyrus, Trifolium, Medicago, Melilotus, and/or Tamarindus.

In particular embodiments the
Phaseolus bean is selected from the species P. vulgaris (Kidney Bean, Pinto Bean, Navy Bean Haricot Bean, Black Beans, Borlotti Beans), P. lunatus (Lima Bean), P. coccineus (Runner Bean, Flat Bean), and/or P. acutifolius (Tepary Bean));
Pisum bean is selected from P. sativum peas (Green Peas, White Peas, Yellow Peas, Field Peas, Snow Peas, Snap Peas);
Vigna bean is selected from V. radiata (Mung Bean), V. mungo (Urad), V. unguiculata (Cowpea, Yardlong bean, Black-eyed Pea), V. aconitifolia (Moth bean), and/or V. angularis (Adzuki bean);
Cajanus bean is selected from C. cajan (Pigeon Pea);
Lens bean is selected from L. culinaris (Lentil, Red Lentil, Green Lentil, Puy Lentil);
Cicer bean is selected from C. arietinum (Chickpea, Garbanzo Bean);
Vicia bean is selected from V. faba (Fava Bean, Broad Bean), V. ervilia (Bitter vetch), and/or V. sativa (common vetch);
Arachis bean is selected from A. hypogaea (peanut);
Glycine bean is selected from G. max (soybean);
Macrotyloma bean is selected from M. uniflorum (Horsegram);
Mucuna bean is selected from M. pruriens (velvet bean);
Lupinus bean is selected from L. albus (white lupin, sweet lupin), L. mutabilis (Tarwi/Andean Lupin), L. hirsutus and/or L. angustifolius;
Ceratonia the bean is selected from C. siliqua (Carob bean);
Canavalia bean is selected from C. gladiate (Sword bean), and/or C. ensiformis (Jack bean);
Cyamopsis bean is selected from C. tetragonoloba (Guar bean);
Lablab bean is selected from L. purpureus (Hyacinth Bean, lablab bean);
Psophocarpus bean is selected from P. tetranoglobulus (winged bean);
Clitoria bean is selected from C. ternatea (butterfly pea);
Lathyrus bean is selected from L. sativus (grass pea) and/or L. tuberosus (tuberous pea);
Trifolium bean is selected from T. repens (white Clover), and/or T. pratense (red clover);
Medicago bean is selected from M. sativa (alfalfa);
Melilotus bean is selected from M. officinalis (sweet clover); and/or
Tamarindus bean is selected from T. indica (tamarind);

The one or more edible ingredients can suitably comprise 5% wt to 60% wt of legumes, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt, optionally 38% wt to 42% wt. Additionally or alternatively, the legume can comprise 5% wt to 60% wt of protein, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt. Additionally or alternatively the legume can comprise 5% wt to 60% wt of starch, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt. The one or more edible ingredients can also comprise at least two legumes, of which at least one legume contains a high level of protein, while at least one other legume contains high levels of starch. More specifically the high-protein level legume can comprise 5% wt to 60% wt of protein, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt, whereas the high starch level legume can comprise 5% wt to 60% wt of protein, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt. In desired embodiments the legume comprises Pisum beans and/or Lupinus beans, particularly P. sativum peas and L. albus lupin seeds, more particularly yellow split P. sativum peas and sweet L. albus lupin seeds. In further useful embodiments the one or more edible ingredients comprise 10% wt to 30% wt, optionally 15% wt to 25% wt, optionally 19% wt to 21% wt of yellow split P. sativum peas. Alternatively, the one or more edible ingredients comprise 15% wt to 25% wt, optionally 19% wt to 21% wt of sweet L. albus lupin seeds. Further, the one or more edible ingredients comprise both 10% wt to 30% wt, optionally 15% wt to 25% wt, optionally 19% wt to 21% wt of yellow split P. sativum peas and 10% wt to 30% wt, optionally 15% wt to 25% wt, optionally 19% wt to 21% wt of sweet L. albus lupin seeds.

In a preferred embodiment the one or more edible ingredients comprise oats, Solanum tuberosum, B. vulgaris var. conditiva, P. sativum peas and L. albus lupin seeds more specifically 10% wt to 30% wt, optionally 15% wt to 25% wt, optionally 19% wt to 21% wt each of oats, potato, red beet, P. sativum peas and L. albus lupin seeds.

For further improvement of properties, the edible ingredients further can also comprise plant extract, such as extracts of oats, optionally aqueous extracts also known as oats milk. In addition, or alternatively the extract may be an almon extract, optionally an aqueous extract of almonds also known as almonds milk. Such aqueous extract can suitably be leftovers from actual production of plant extracts, such as the press cake from such extract production.

### Preparation of Edible Ingredients

Prior to fermentation, the edible ingredients are advantageously pre-processed in a manner improving the conditions for subsequent handling and/or fermentation. As disclosed herein, it has been found that the preprocessing of ingredients significantly influences the effect of fermentation and the quality parameters of the fermented product. In some embodiments, the preprocessing is mechanical and includes chopping, grinding, and/or shredding, while in other embodiments the preprocessing is thermal or chemical, such as heating, microwaving, boiling and/or steaming, especially prior to forming the fermentation substrate.

In some embodiments cereals are milled or ground to a particle size having a maximum diagonal/diameter of 0.5 mm to 10 mm, such as 0.5 mm to 1 mm, such as 1 mm to 2 mm, such as 2 mm to 3 mm, such as 3 to 4 mm, such as 4 mm to 5 mm, such as 5 mm to 6 mm, such as 6 mm to 7 mm, such as 7 mm to 8 mm, such as 8 mm to 9 mm, such as 9 mm to 10 mm, optionally to an average particle size of 3.5 mm. In particular, where the cereal is oats and/or BSG, the oats and/or BSG, before being formed into the fermentation substrate, undergo pre-processing by milling them into particles ranging between 2 and 5 mm in size, optionally between 3 and 4 mm, and optionally to an average particle size of 3.5 mm.

In other embodiments the one or more edible ingredients are pre-processed, particularly prior to forming the fermentation substrate, by heat, for example by microwaving, boiling, or steaming, to gelatinize among others starch components. For the fermented product described herein steaming is particularly useful, as it provides some water for gelatinization while still controlling the amount of water in the substrate prior to fermentation. Steaming leads to a more precise cooking, while e.g. boiling would remove nutrients and increase the water content in the substrate.

More specifically, tubers such as potatoes or other high-starch containing ingredients can be advantageously pre-processed through heat treatment to expose starch granules for the fermenting microorganism and to gelatinize the starch fully or partially through methods such as microwaving, boiling, or steaming. Where the ingredients include both cereals and root vegetables, particularly oats and potatoes, these can also be co-ground prior to forming the fermentation substrate, optionally to a degree where no visible chunks of the root vegetable can be observed. In some embodiments, in particular where the cereal is oats and the root vegetable is potato, the edible ingredients include co-ground cereal and root vegetable, preferably in a ratio of 0.5:1 to 1:0,5 of cereal:root vegetable, optionally 1:1. In some embodiments cereal:root vegetable ratio is oats:potato.

In still further embodiments of pre-processing where the root vegetable is a taproot vegetable such as Beta vulgaris, optionally subspecies vulgaris, in particular B. vulgaris var. conditiva (red beet), the tap root is pre-processed by i) boiling and/or steaming the tap root and ii) grinding the tap root prior to forming the fermentation substrate. In this embodiment the taproot is suitably ground to an average particle size in its longest diagonal/diameter as described, supra, preferably between 1.7 mm to 2.8 mm.

In the method described herein the one or more edible ingredients comprise P. sativum peas, and wherein the P. sativum peas, prior to forming the fermentation substrate, is pre-processed by i) drying and splitting the P. sativum peas, ii) microwaving, boiling, or steaming the P. sativum peas and iii) grinding the P. sativum peas to an average particle size as described, supra, preferably between 1,7-2.8 mm.

Where the edible ingredients include legumes, these are preferably also pre-processed, prior to forming the fermentation substrate to both gelatinize and open the ingredient components to the microorganism and to remove undesired components such as toxic and/or bitter tasting compounds, such as alkaloids. This pre-processing suitably includes soaking the legume is an aqueous liquid to extract undesired compounds to microwave, boil or steam the legume and to grind the legume. For example, where the legume is beans/seeds of Lupinus, such as from L. albus (white lupin, sweet lupin), L. mutabilis (Tarwi/Andean Lupin), L. hirsutus and/or L. angustifolius, in particular from L. albus; these can advantageously be soaked in fresh or salted water to wholly or partially extract toxic or bitter tasting alkaloids and ground to an average particle size in the longest diagonal/diameter as described, supra, preferably between 1.7 mm to 2.8 mm.

In a further embodiment the substrate is prepared from red beet, potato, oatmeal, split peas, and lupin beans in an even mix of 20% wt. Red beets and potatoes are preferably peeled, cut, and steamed, oatmeal is preferably ground to a flour, split peas are preferably were boiled, and lupins are preferably soaked for removing undesirable components (such as at least for 6 hours) before boiling. Red beets, potatoes, and lupins are preferably blended separately to increase the surface area. The potatoes are preferably blended with the oatmeal flour in a 1:1 ratio (W/W) to decrease stickiness.

### Fermentation Substrate

In preferred embodiments the method described herein included a step of forming the edible ingredients, whether pre-processed or not, into a fermentation substrate. This step suitably includes operations selected from further mechanical, ultrasound and/or thermal treatment optionally as well as addition processing aids or other functional ingredients.

Mechanical steps include mixing of ingredients, further grinding of the mixtures, and/or shaping the mixtures into a desired shape suitable for inoculation by the microorganism and for the fermentation process. Thermal treatment can also include additional steps such as microwaving, boiling, or steaming of the ingredient mixtures, including procedures to sanitize the formed substrate, such as pasteurization.

In some embodiment the substrate forming step includes mixing the ingredients, whether pre-processed or not, and optionally further grinding them to an average particle size in the longest diagonal/diameter of between 50 μm to 500 μm, such as between 50 μm to 100 μm, such as between 100 μm to 150 μm, such as between 150 μm to 200 μm, such as between 200 μm to 250 μm, such as between 250 μm to 300 μm, such as between 300 μm to 350 μm, such as between 350 μm to 400 μm, such as between 400 μm to 450 μm, such as between 450 μm to 500 μm,. In other embodiments the substrate forming step include grinding ingredients to an average particle size in the longest diagonal/diameter of between 0.5 to 10 mm, such as 0.5 to 1 mm, such as 1 to 2 mm, such as 2 to 3 mm, such as 3 to 4 mm, such as 4 to 5 mm, such as 5 to 6 mm, such as 6 to 7 mm, such as 7 to 8 mm, such as 8 to 9 mm, such as 9 to 10 mm. Optionally the substrate forming step includes pasteurizing the mixture before and/or after grinding. This approach is particularly useful when the ingredients include oat, potato, red beet, P. sativum peas and L. albus lupin beans.

Pasteurization of the substrate prior to inoculation and fermentation is important to avoid contamination of the substrate with microorganisms which could negatively impact on the fermentation. Pasteurization of substrates can typically be accomplished by steaming at 120° C. for 30 minutes.

In important embodiments, the water content of the substrate is also adjusted prior to fermentation where the best fermentation performance can be achieved with a moisture content of between 25% wt to 50% wt, such as between 25% wt to 30% wt, such as between 30% wt to 35% wt, such as between 35% wt to 36% wt, such as between 36% wt to 38% wt, such as between 38% wt to 39% wt, such as between 39% wt to 41% wt, such as between 41% wt to 43% wt, such as between 43% wt to 45% wt, such as between 45% wt to 50% wt.

Other important embodiments include those in which the substrate is divided and formed into a number of discrete portions with a desired size and/or shape, and then placed into a container optimized for the subsequent fermentation and/or final use of the fermented product. Preferred portion sizes are between 25 g to 3000 g, such from 25 g to 50 g, such from 50 g to 100 g, such from 100 g to 200 g, such from 200 g to 300 g, such from 300 g to 400 g, such from 400 g to 500 g, such from 500 g to 750 g, such from 750 g to 1000 g, such from 1000 g to 1500 g, such from 1500 g to 2000 g, such from 2000 g to 2500 g, such from 2500 g to 3000 g. The portions are formed into desired shapes, such as granules, balls, blocks, sheets, or patties, and then placed into containers that are useful for maintaining both the shape and the fermentation process. Such containers may be of open or closed design for optimal fermentation results and/or handling, and they may be made from wood, plastic, metal, or composites thereof.

### Microorganisms and Fermentation

The method described herein also includes the step of contacting the fermentation substrate with one or more microorganisms under conditions allowing the microorganisms to colonize and proliferate on/in the fermentation substrate. The contacting includes any suitable way of introducing or inoculating the microorganisms onto or into the substrate, include the mixing into the substrate or spraying or dusting onto a surface of the substrate of the microorganism. The microorganism can be added to the substrate as a lyophilized powder or as a pre-inoculation cell suspension, preferably in amounts of 0.25 g to 2 g of microorganism per kilogram substrate. In some embodiments the substrate is inoculated with between 0,01 g to 0,05 g koji spores per kg substrate, such as between 0,02 g to 0,04 g, such as about 0.033 g koji spores per kg. substrate.

The microorganism used in the method described herein is preferably a GRAS (Generally Recognized As Safe) strain, which is particularly useful in the sense that less safety studies are required for the fermented edible product to be market approved. Further, in some embodiments the microorganism is a fungus, such as a filamentous fungus or mould, optionally of the genus Aspergillus, optionally of the species Aspergillus oryzae or Aspergillus sojae. Other preferred moulds include those of the genus Rhizopus optionally of the species Rhizopus oligosporus. Useful subvariants of A. oryzae include A. Oryzae “sweet koji” and A. Oryzae “protein koji”.

The substrate inoculated with the microorganism(s) is allowed to ferment under conditions allowing the microorganisms to colonize and proliferate on/in the fermentation substrate preferably during a predetermined time interval, temperature, and humidity to achieve optimal improvement of the properties. In preferred embodiments the fermentation time is selected from between 20 hours to 90 hours, such as between 20 hours to 30 hours, such as between 30 hours to 40 hours, such as between 40 hours to 50 hours, such as between 50 hours to 60 hours, such as between 60 hours to 70 hours, such as between 70 hours to 80 hours, such as between 80 hours to 90 hours. In preferred embodiments the fermentation temperature is selected from between 10° C. to 70° C., such as from 10° C. to 19° C., such as from 19° C. to 20° C., such as from 20° C. to 25° C., such as from 25° C. to 30° C., such as from 30° C. to 31° C., such as from 31° C. to 35° C., such as from 35° C. to 40° C., such as from 40° C. to 50° C., such as from 50° C. to 60° C., such as from 60° C. to 70° C. To stop fermentation, the substrate and microorganism are suitably cooled, preferably to below 10° C., and subsequently pasteurized at 90° C. for 3 minutes to heat inactivate any remaining microbial activity.

During fermentation further moisture can be supplied, to wholly or partially maintain a moisture level optimal for the fermentation. However, the fermentation process may still spend moisture and the fermented product may require rehydration to maintain attractive properties. Accordingly, in one embodiment the fermented product is rehydrated by the adding water to the product to reach a moisture content between 20% wt to 90% wt, such as 20% wt to 30% wt, such as 30% wt to 40% wt, such as 40% wt to 50% wt, such as 50% wt to 60% wt, such as 60% wt to 70% wt, such as 70% wt to 80% wt, such as 80% wt to 90% wt.

### Parameters

The method described herein improves or enriches the fermented product in one or more properties selected from flavour, aroma, texture, cooking experience, visual appearance, nutritional and/or dietary value, spoilage time, and/or carbon footprint compared to a parent unfermented product. Such properties are, if not directly quantifiable by analytical methods, usually assessed and determined by one or more panels of seasoned and experienced food scientists and/or chefs.

Improved flavour properties include enhancements in selected properties such as sourness, sweetness, bitterness, saltiness, umami, or a combination thereof, compared to the unfermented parent product. Improved aroma properties include improvements or enhancements in the aromas of raw ingredients, characterized by a strong vegetal and bean aroma, while fermented products exhibit milder aromas of toasted cereal, sweet porridge, stone fruit, and mushroom.

Improved texture properties include improvements in properties selected from firmness, softness, cohesiveness, juiciness, chewiness, sandiness, or a combination thereof compared to a parent unfermented product. Improved cooking properties include improvements in (a) browning properties of the fermented edible product (Maillard reaction) when roasting or frying the fermented edible product compared to roasting or frying a parent unfermented product, and/or (b) structure and integrity of the fermented product by the microorganism biomass binding together the edible ingredients compared to a parent unfermented product. Improved visual properties include improvement in colour of the fermented product, compared to the colour of a parent unfermented product. Improved nutritional properties include improvements in content and/or distribution of proteins, lipids, carbohydrates, fibres, vitamins, minerals, amino acids, or a combination thereof compared to a parent unfermented product. Still further improved spoilage time includes improvement in the shelf-life of the fermented product. This applies to both ambient and cooled conditions, as well as to packaged and unwrapped conditions, resulting from the microorganisms' control and competition effects on other microbial fauna in the fermented product. Improved carbon footprint follows and results not only from the improved or extended spoilage time, but also from the inclusion of ingredients sourced from industrial waste streams. This reduces or lowers the need and/or demand for new (virgin) agricultural produce, thereby decreasing and/or reducing both fossil fuel consumption during crop cultivation and the utilization of land areas.

### Product

The methods described herein produce unique edible products with improved properties. Additionally, as a separate aspect described herein, a fermented edible product is provided, which is produced using the methods disclosed herein. The product preferably comprises one or more filamentous fungi or the mycelium thereof and 10% wt to 30% wt, optionally 15% wt to 25% wt, optionally 19% wt to 21% wt each of cereal, root vegetable, and legume, optionally 10% wt to 30% wt, optionally 1 5% wt to 25% wt, optionally 19% wt to 21% wt each of oats, potato, red beet, P. sativum peas and L. albus lupin seeds. In a further embodiment the fermented edible product comprises more than 5% wt of dietary fibres, such as between 5% wt to 20% wt, such as 5% wt to 10% wt, such as 10% wt to 15% wt, such as 15% wt to 20% wt.

### EXAMPLES

The following non-limiting examples illustrate the methods and products of this disclosure.

### Example 1—Testing Impact of Cereal Grain Ingredients on Texture and Flavour

A range of cereal grain preparations were tested to identify the best performing dry component for the fermentation, to improve the properties of the fermented product.

Each sample was prepared by mixing a test cereal with pureed potato until a uniform and comparable texture was achieved. The mixture was adjusted to a suitable hydration level for fungal growth during fermentation, as indicated in Table 1. After mixing the ingredients, each composition was mixed with 0,05% wt of lyophilized Aspergillus oryzae/Aspergillus sojae available from a commercial vendor by sprinkling via a tea strainer. 75-gram samples of each composition were placed in Ø122 mm plastic open gastro trays. The sample trays were then placed in a temperature and humidity-controlled incubator and incubated for 40 Hours at 30° C. with 75% relative humidity.

TABLE 1

| Sample # | Sample Composition |
|---|---|
| 1 | Oat flour:potato - 1:1 |
| 2 | Toasted wheat flour:potato - 1:1 |
| 3 | Barley flour:potato - 1.1:1 |
| 4 | Rye flour:potato - 1.5:1 |
| 5 | Millet flakes:potato - 1:2 |
| 6 | Sorghum flour:potato - 2:1.5 |
| 7 | Red lentil flour:potato - 1.5:1 |
| 8 | Malted barley flour (sifted):potato - 1.5:1 |
| 9 | Rye flour:potato - 2:1 |
| 10 | Bulgur flour:potato - 2:1 |
| 11 | Blended rye flakes:potato - 2:1 |

### Results/Conclusion

The most promising results were observed in sample 2 and sample 10, which exhibited attractive characteristics in terms of both flavour and texture. These characteristics included a chewy, bouncy texture that resembled the texture of meat. Further, samples 1 and 2 had a mild flavour, that could easily be customized at a later step, whereas samples 5 and 6 had a stronger grain or cereal flavour, but a very meaty, firm texture. Sample 10 had a different texture that was less meaty but still firm. Samples 5 and 6 had a firm bite that disintegrated and became mushy. Sample 2 had small particles with some chew and bite, which created a longer chewing experience.

### Example 2—Testing Legume Ingredients Impact on Texture and Flavour/Aroma

A range of legumes were tested to identify best performing legume for fermentation to improve the properties of the fermented product. Test ingredients were pre-processed as follows:
a) Oats was milled and sifted using a 3.5 mm hole size, followed by toasting at 90° C. for 3 min.
b) Potatoes were and stored in water for 3 days, followed by peeling and cutting into 3×3 cm cubes. Then the cubes were steamed for 20 min at 100° C. and then blast chilled to 5° C.
c) Oats and potatoes were then ground together.
d) Beets were washed, peeled, and stored in water for 3 days, then cut into 3×3 cm cubes, steamed for 40 min at 100° C., and then ground to a particle size of less than 3 mm.
e) Split peas were boiled in water for 24 min, drained and air dried for 10 min and then ground to a particle size of less than 3 mm.
f) Lupin beans were soaked in water overnight and repeatedly rinsed in water, then boiled in water for 40 min drained and air dried for 10 min and then ground to a particle size of less than 3 mm.

Samples were prepared by mixing ingredients according to table 2. [Method of preparation] After mixing the ingredients each composition was mixed with lyophilized Aspergillus oryzae/Aspergillus sojae from a commercial vendor, by sprinkling via a tea strainer. 75 gram samples of each composition were placed in Ø122 mm plastic open gastro trays (*sample 258 was a large sample of 1501 grams). The sample trays were then placed in a temperature and humidity-controlled incubator and incubated at 30° C. for 43 hours at 75% relative humidity.

TABLE 2

| | | Root vegetable | | | Legume | | | | | | | | |
|---|---|---|---|---|---|---|---|---|---|---|---|---|---|
| | Cereal | % wt | | | % wt | | | | | | | | |
| Sample | % wt | | Red | Yellow | | Ingrid | | | | | | Birgit | Broad |
| # | BSG | Potato | Beets | Beets | Lupin | Split Pea | Frida | Sirius | Bourgina | Probor | Ingrid | Beans | bean |
| 258* | 19 | 21 | 20 | — | 20 | 20 | — | — | — | — | — | — | — |
| 259 | 19 | 21 | 20 | — | 20 | 20 | — | — | — | — | — | — | — |
| 260 | 19 | 21 | 20 | — | 20 | 20 | — | — | — | — | — | — | — |
| 261 | 19 | 21 | — | 20 | 20 | 20 | — | — | — | — | — | — | — |
| 262 | 19 | 21 | 20 | — | — | — | — | — | — | — | — | — | 40 |
| 263 | 19 | 19 | 27 | — | 16 | 20 | — | — | — | — | — | — | — |
| 264 | 19 | 21 | 20 | — | 20 | 20 | — | — | — | — | — | — | — |
| 265 | 19 | 21 | 20 | — | — | — | — | — | — | — | — | 40 | — |
| 266 | 19 | 21 | 20 | — | — | — | — | — | — | — | — | 40 | — |
| 267 | 19 | 21 | 20 | — | — | — | — | — | — | — | — | 40 | — |
| 268 | 19 | 21 | 20 | — | — | — | 40 | — | — | — | — | — | — |
| 269 | 19 | 21 | 20 | — | — | — | — | 40 | — | — | — | — | — |
| 270 | 19 | 21 | 20 | — | — | — | — | — | 40 | — | — | — | — |
| 271 | 19 | 21 | 20 | — | — | — | — | — | — | 40 | — | — | — |
| 272 | 19 | 21 | 20 | — | — | — | — | — | — | 40 | — | — | — |

### Results/Conclusion

After fermentation/incubation, all the tested legumes exhibited varying degrees of desirable texture characteristics. Some legumes resulted in a softer and more “wet bread” texture, offering a greater level of chewiness and bounce compared to others. Sample 258 had a superior texture over samples 267, 268, 269, 270, 271, 272, indicating that the split pea and lupin mixture is the superior combination of legumes as it provides the most favourable organoleptic results. Flavour varied even more drastically between the tested legumes, i.e., sample 263 absorbed more water, resulting in a slight damp or musky aroma-indicating the impact of hydration on flavour/aroma.

Selected sample observations:

### Sample 263 (Contaminated):

Flavour/aroma: musky, bathroom, mould, damp smell.

### Sample 261:

Texture: complete porridge texture, very soft, humid, and mushy/wet
Flavour/aroma: musky aromas, sourness, desirable Maillard browning.

Overall, not pleasant texture or flavour/aroma, but not ruling out golden beets as a possible ingredient.

### Sample 264:

Texture: very soft texture, very humid, limited bite or chew, wet bread texture
Flavour/aroma: slight hints of musk, present bitterness, umami taste, raw bean and potato flavour, very little conversion, lacking mycelium.

### Sample 267:

Texture: wet bread, no bite, not very chewy, porridge
Flavour/aroma: lacking umami, some muskiness, not pleasant

### Example 3—Testing Microorganism Impact on Flavour/Aroma

A range of microorganism preparations were tested to identify best performing GRAS filamentous fungi or combination of microorganisms for fermentation and for improving the properties of the fermented product. BSG, potato, red beet, P. sativum peas and L. albus lupin seeds were pre-processed as follows:

Substrate was prepared from red beet, potato, BSG, split peas, and lupin beans in an even mix of 20 wt % of the ingredient. Red beets and potatoes were peeled, cut, and steamed, BSG was ground to a flour, split peas were boiled, and lupins were soaked for a minimum of 6 hours before boiling. Red beets, potatoes, and lupins were then blended separately to increase the surface area. The potatoes were blended with the BSG flour in a 1:1 ratio (W/W) to decrease “stickiness”. Ingredients were then mixed together to a substrate in a churning drum where it, prior to inoculation, was pasteurized by steaming at 120° C. for 30 minutes. After the steaming the substrate was allowed to cool to below 35° C. Samples of the substrate were drawn and inoculated with different microorganisms from a commercial vendor (see table 3) and incubated as in example 2.

TABLE 3

| Sample # | Description | Container | Sample size grams | |
|---|---|---|---|---|
| 1 | Aspergillus | White | 1500 | g |
| | Oryzae type 1 | Proofing | | |
| | | box | | |
| 2 | Aspergillus | 1.5 kg | 1500 | g |
| | Oryzae type 1 | gastro | | |
| 3 | Aspergillus | 1.5 kg | ?? 1500 | g |
| | Oryzae type 1 | gastro | | |
| 4 | Aspergillus | 1.5 kg | ?? 1500 | g |
| | Oryzae type 2 | gastro | | |
| 5 | Rhizopus | 1.5 kg | ?? 1500 | g |
| | oligosporus | gastro | | |
| 6 | | 350 g | ?? 350 | g |
| | | (1.5 L | | |
| | | condi) | | |
| 7 | Aspergillus | 350 g | ?? 350 | g |
| | Oryzae type 3 | (1.5 L | | |
| | | condi) | | |

### Results/Conclusion

After 24 hours, the best growth was achieved in samples 1 to 3. Sample 6 produced a very pleasant, sweet aroma, indicating a healthy fermentation, while in sample 4 fermentation progressed very slowly (no detectable aromas, and cold internal temperature).

### Example 4—Testing Oats, Wheat, and Bulgur as Cereals

A range of ingredient combinations of cereals—oats, wheat and bulgur were tested to identify best performing cereal for fermentation and for improving the properties of the fermented product.

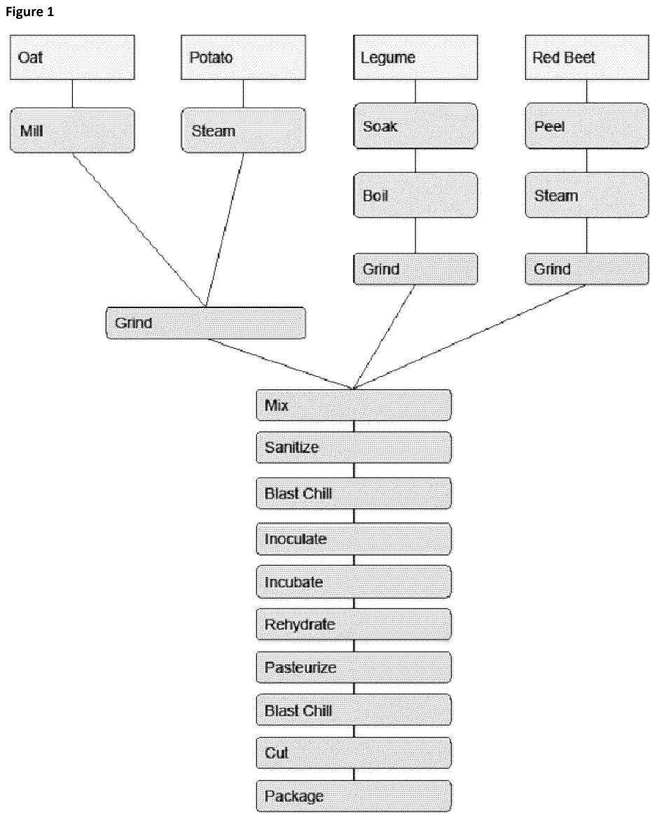

The ingredients for the experiments were pre-processed as follows (see also FIG. 1):
a) Potatoes were peeled, cut in 3 cm×3 cm cubes and steamed in an oven for 15 minutes and thereafter blast chilled and placed in refrigerator for later use.
b) Red beets were peeled, cut in cubes and steamed in an oven for 45 minutes and thereafter blast chilled. Once cooled, the red beet was transferred a to Robot Coupe Food Processor and ground to particle size 1,7-2.8 mm.
c) Peas were boiled for 10 minutes, cooled and airdried and transferred to a Robot Coupe Food Processor and ground to particle size 1,7-2.8 mm.
d) Lupin seeds/beans were soaked in water overnight, boiled for 20 minutes, allowed to airdry and transferred to a Robot Coupe Food Processor and ground to particle size 1,7-2.8 mm.
e) Steel-cut oats were milled using a Hawos Billy 100 grinder to an average particle size of 3.5 mm.
f) Wheat berries were milled using a Hawos Billy 100 grinder to an average particle size of 3.5 mm.
g) Bulgur was milled using a Hawos Billy 100 grinder to an average particle size of 3.5 mm

Further, where grains were mixed with potato, the grain and steamed potato were ground for 1 minute in a Robot Coupe Food Processor until no potato chunks are visible and clumps begin to hold together. This pre-processing resulted on the mixtures of table 4.

TABLE 4

| | | No. of |
|---|---|---|
| Mixture # | Composition | samples drawn |
| 1 | 50% wt milled oats | 12 |
| | 50% wt potato | |
| 2 | 50% wt unmilled oats | 1 |
| | 50% wt potato | |
| 3 | 50% wt milled wheat berries | 1 |
| | 50% wt potato | |
| 4 | 67% wt milled bulgur | 1 |
| | 33% wt potato | |
| 5 | 25 g % wt milled oats | 1 |
| | 33% wt milled bulgur | |
| | 42% wt potato | |
| 6 | 50% wt milled oats | 1 |
| | 50% wt split pea | |
| 7 | 50% wt milled oats | 1 |
| | 50% wt lupin seed/bean | |

The mixtures were prepared in a churning drum where it, prior to inoculation, was pasteurized by steaming at 120° C. for 30 minutes. After the steaming the mixtures were allowed to cool to 35° C. after which the mixtures were inoculated with 0.033 g koji spores per kg substrate allowing the churning drum to churn for 5 minutes. 75-gram samples were drawn and placed in containers. The containers were placed in a temperature and humidity-controlled incubator and incubated for 43 hours at 30° C. and 75% relative humidity.

### Results/Conclusions:

From the seven mixtures, 1, 2 and 3 were the most successful in terms of texture. Oat, both milled and unmilled had very favourable texture that had great bite and chew. Mixture 3 (wheat) also had a high chew and meat-like texture, but a more pronounced grain aftertaste. All mixtures resulted in a successful incubation while oats and wheat were most favourable in terms of texture and flavour.

### Example 5—Ingredient Combinations

A range of ingredient combinations were tested to identify best performing combination for fermentation and for improving the properties of the fermented product. The ingredients for the experiments were pre-processed as follows (see also FIG. 1):
a) BSG [type] available from a commercial brewery was dehydrated for 48 h at 70° C. and first ground in a thermomixer and then ground in a coffee grinder.
b) Potatoes were peeled, cut in 3 cm×3 cm cubes and steamed in an oven for 15 minutes and thereafter blast chilled and placed in refrigerator for later use.
c) Red beets were peeled, cut in cubes, and steamed in an oven for 45 minutes and thereafter blast chilled. Once cooled, the red beet was transferred a to Robot Coupe Food Processor and ground to particle size 1,7-2.8 mm.
d) Peas were boiled for 10 minutes, cooled and airdried and transferred to a Robot Coupe Food Processor and ground to particle size 1,7-2.8 mm.
e) Lupin seeds/beans were soaked in water overnight, boiled for 20 minutes, allowed to airdry, and transferred to a Robot Coupe Food Processor and ground to particle size 1,7-2.8 mm.
f) Steel-cut oats were milled using a Hawos Billy 100 grinder to an average particle size of 3.5 mm.
g) Wheat berries were milled using a Hawos Billy 100 grinder to an average particle size of 3.5 mm.
h) Dried wheat breadcrumbs were purchased on the market.
i) Dried rye breadcrumbs were purchased on the market and further dried to 25% moisture.

Further, where grains were mixed with potato, the grain and steamed potatoes were ground for 1 minute in a Robot Coupe Food Processor until no potato chunks are visible and clumps begin to hold together. This pre-processing resulted on the mixtures of table 5.

TABLE 5

| | | | No | Sample size |
|---|---|---|---|---|
| Mixture # | Composition | Container | Samples | (gram) |
| 1 | 20 wt % milled oats | Plastic | 1 | 1500 |
| | 20% wt % potato | gastro tray | | |
| | 20% wt % red beet | | | |
| | 20% wt % lupin | | | |
| | seeds/beans | | | |
| | 20% wt % split pea | | | |
| 2 | 20 wt % milled | Metal tray | 4 | 1500 |
| | wheat berries | | | |
| | 20% wt % potato | | | |
| | 20% wt % red beet | | | |
| | 20% wt % lupin | | | |
| | seeds/beans | | | |
| | 20% wt % split pea | | | |
| 3 | 20 wt % milled oats | Metal tray | 4 | 75 |
| | 20% wt % potato | | | |
| | 20% wt % red beet | | | |
| | 20% wt % lupin | | | |
| | seeds/beans | | | |
| | 20% wt % split pea | | | |
| 4 | 20 wt % milled | Metal tray | 4 | 75 |
| | wheat berries | | | |
| | 20% wt % potato | | | |
| | 20% wt % red beet | | | |
| | 20% wt % lupin | | | |
| | seeds/beans | | | |
| | 20% wt % split pea | | | |
| 5 | 20% wheat breadcrumbs, | Metal tray | 4 | 75 |
| | 20% wt % potato | | | |
| | 20% wt % red beet | | | |
| | 20% wt % lupin | | | |
| | seeds/beans | | | |
| | 20% wt % split pea | | | |
| 6 | 20% rye breadcrumbs, | Metal tray | 4 | 75 |
| | 20% wt % potato | | | |
| | 20% wt % red beet | | | |
| | 20% wt % lupin | | | |
| | seeds/beans | | | |
| | 20% wt % split pea | | | |

The mixtures were prepared in a churning drum where it, prior to inoculation, were pasteurized by steaming at 120° C. for 30 minutes. After the steaming the mixtures were allowed to cool to 35° C. after which the mixtures were inoculated with A. oryzae from a commercial vendor allowing the churning drum to churn for 5 minutes and samples were drawn according to table 5 and placed in containers. The containers were placed in a temperature and humidity-controlled incubator and incubated for 43 hours at 30° C. and 75% relative humidity.

### Results/Conclusions

Oat and wheat proved both to produce exceptional results with great bite, chew, and a mild flavour—with oats as the preferred cereal grain. Wheat breadcrumbs also produced a very delicate texture that was more tender and less chewy somewhat resembling the texture of chicken, whereas wheat and oats resembled more ground beef texture. Rye breadcrumbs had a much slower fermentation and less pronounced mycelium, resulting in a lack of strength in texture. It was contemplated that the acidity of rye breadcrumbs inhibited the growth of fungus.

### Example 6—Impact of Substrate Density on Quality Properties

The main objective of this experiment was to evaluate the effect of substrate density, on weight loss and other properties of the fermented substrate. For this, the diameter and weight of substrate patties were fixed while the patty thickness was modified by pressing or non-pressing the substrate to different thickness. It is hypothesized that higher densities will affect the fermentation by decreasing the intra-product oxygen/CO₂ transfer. Additionally, a difference in density is hypothesized to affect the sensory profile of the fermented substrate.

### Equipment Used

Metal patty rings (18.8 cm in diameter and 2.8 cm in height) for forming substrate patties Precision scale.

### Protocol

Substrate was prepared from red beet, potato, oatmeal, split peas, and lupin beans in an even mix of 20 wt % of the ingredient. Red beets and potatoes were peeled, cut, and steamed, oatmeal was ground to a flour, split peas were boiled, and lupins were soaked for a minimum of 6 hours before boiling. Red beets, potatoes, and lupins were then blended separately to increase the surface area. The potatoes were blended with the oatmeal flour in a 1:1 ratio (W/W) to decrease “stickiness”. Ingredients were then mixed together to a substrate in a churning drum where it, prior to inoculation, was pasteurized by steaming at 120° C. for 30 minutes. After the steaming the substrate was allowed to cool to below 35° C. after which the substrate was inoculated with 0.033 g koji spores per kg substrate in the churning drum and churned for a further 5 minutes. Then the substrate was then distributed into the patty rings and subjected to the different density modifications. Then the substrate patties with different densities were placed in a temperature and humidity-controlled incubator and incubated for 43 hours at 30° C. and 75% relative humidity.

### Results

Before Fermentation

| | | Initial | | | Dry | |
|---|---|---|---|---|---|---|
| Patty | Diameter | height | Volume | Weight | weight | Density |
| Number | (cm) | (cm) | (cm³) | (g) | (g) | (g/cm³) |
| 1 | 12.8 | 2.8 | 360.3029783 | 172 | 78.3 | 0.477376015 |
| 2 | 12.8 | 2.3 | 295.9631607 | 172 | 78.3 | 0.58115341 |
| 3 | 12.8 | 2 | 257.3592702 | 172 | 78.3 | 0.668326421 |
| 4 | 12.8 | 1.7 | 218.7553797 | 172 | 78.3 | 0.668326421 |

After Fermentation

| Patty | Diameter | Height | Volume | Weight | Dry weight | Density |
|---|---|---|---|---|---|---|
| Number | (cm) | (cm) | (cm³) | (g) | (g) | (g/cm³) |
| 1 | 10.5 | 2.2 | 190.4983245 | 96.84 | 59.88101773 | 0.508350928 |
| 2 | 10.4 | 2 | 169.8973307 | 97.21 | 59.81410537 | 0.572169084 |
| 3 | 10.5 | 1.7 | 147.2032508 | 96.09 | 60.4130198 | 0.65277091 |
| 4 | 10.6 | 1.6 | 141.1957402 | 96.2 | 59.90172414 | 0.681323671 |

Analysis

| Patty | Diameter | Diameter | Thickness | Thickness | Weight | Weight loss |
|---|---|---|---|---|---|---|
| Number | difference (cm) | decrement | difference (cm) | decrement (%) | Loss (g) | ratio (%) |
| 1 | 2.3 | 18% | 0.6 | 21% | 75.16 | 44% |
| 2 | 2.4 | 19% | 0.3 | 13% | 74.79 | 43% |
| 3 | 2.3 | 18% | 0.3 | 15% | 75.91 | 44% |
| 4 | 2.2 | 17% | 0.1 | 6% | 75.8 | 44% |

Analysis

| | | Volume loss | Dry Weight | | Density loss | |
|---|---|---|---|---|---|---|
| Patty | Volume loss | ratio | loss | Density diff. | ratio | Chewiness |
| Number | (cm³) | (%) | (g/g) | (g/cm³) | (%) | (1 < 5) |
| 1 | 169.8046537 | 47% | 0.24 | −0.030974913 | −6.5% | 4 |
| 2 | 126.06583 | 43% | 0.24 | 0.008984326 | 1.5% | 3 |
| 3 | 110.1560194 | 43% | 0.23 | 0.015555511 | 2.3% | 2 |
| 4 | 77.55963943 | 35% | 0.24 | −0.012997249 | −1.9% | 1 |

| | Before fermentation | | Fermentation indicators | | | |
|---|---|---|---|---|---|---|
| | | Porosity | Weight | Volume | Dry | |
| Patty | Density | (cm³air / cm³ | loss | loss | Weight | |
| Number | (g/cm³) | whole matrix) | ratio (%) | ratio (%) | loss (g/g) | Chewiness |
| 1 | 47.74% | 39.30% | 44% | 47% | 0.24 | 4 |
| 2 | 58.12% | 26.10% | 43% | 43% | 0.24 | 3 |
| 3 | 66.83% | 15.00% | 44% | 43% | 0.23 | 2 |
| 4 | 66.83% | 0.00% | 44% | 35% | 0.24 | 1 |

### Conclusion

Surprisingly the weight loss was almost the same in all samples. However, the relative volume loss decreased more in higher densities. Additionally, it is proved that higher densities have higher final chewiness than more dense products. It seems that a change in density did not have any effect in final weight, dry weight loss (indicating that fermentation performance was similar in all patties) and water loss. However, it is evident that initial substrate density has a direct effect on final product texture.

### Example 7—Impact of Substrate Water Content on Quality Properties

The main objective of this experiment was to evaluate the effect of moisture contents on the preprocessing of the substrate and the properties of the fermented substrate.

### Equipment Used:

Water spray bottle, precision scale.

### Protocol

A substrate was prepared according to example 6. Two samples of the substrate were drawn. One was kept as reference and water was added to other samples to modify its water content. The substrate samples were then inoculated and fermented as described in example 6.

### Results

| | Reference | Sample 1 | Sample 2 | Sample 3 |
|---|---|---|---|---|
| Weight Container | 9.08 | 9.11 | 9.05 | 9.09 |
| Weight Container + | 23.21 | 30.51 | 31.6 | 32.1 |
| substrate | | | | |
| Weight Container + | 15.06 | 15.00 | 15.01 | 15.01 |
| dried substrate | | | | |
| DW | 0.42 | 0.28 | 0.28 | 0.29 |
| Water content | 0.58 | 0.72 | 0.74 | 0.76 |

### Conclusion

It was observed that during the mixing of the first sample, that an equal particle size distribution as the combination of the particles sizes of the ingredients was achieved. For the second sample, larger particle sizes (>1 cm) were observed due to an agglomeration of the substrate. The creation of larger particle sizes caused the production of unfermented spots inside the bigger particles. It was observed that the mycelium could not reach the centre of big particle sizes causing a non-pleasant smells.

1. A method for preparing a fermented edible product enriched or improved in one or more properties selected from flavour, aroma, texture, cooking experience, visual appearance, nutritional and/or dietary value, spoilage time, and/or carbon footprint comprising:
a) Providing one or more edible ingredients;
b) subjecting the one or more edible ingredients to one or more preparation steps to form a fermentation substrate;
c) contacting the fermentation substrate with one or more microorganisms under conditions allowing the microorganisms to colonize and proliferate on/in the fermentation substrate; and
d) isolating the fermentation substrate and the one or more microorganisms into the edible product.

2. The method of claim 1, wherein the one or more of the edible ingredients are or are comprised in an industrial waste product, not used for human consumption.

3. The method of any preceding claim wherein the one or more edible ingredients comprise one or more of:
a) a dry component comprising i) starch providing nutrient to the one or more microorganism and
ii) fibres providing nutritional and/or dietary value to the edible product, the dry component being capable of absorbing moisture from other edible ingredients;
b) a high-moisture and high starch component providing for formation of gelatinized starch binding other edible ingredients together and providing nutrient to the one or more microorganism as well as texture to the edible product;
c) a component providing minerals and vitamins for the one or more microorganism as well as a natural colour to the edible product;
d) a high-protein and low moisture component providing flavour and firmness and structure to the texture of the edible product; or
e) High starch component providing sustained nutrients for the one or more microorganism and providing softness to the texture of the edible product.

4. The method of any preceding claim wherein the one or more edible ingredients comprise at least 2, such as at least 3, such as at least 4, such as at least 5 components selected from the dry component, the high-moisture and high starch component, the component providing minerals and vitamins, the high-protein and low moisture component, or the high starch component.

5. The method of any preceding claim wherein the one or more edible ingredients comprise 5 ingredients selected from the dry component, the high-moisture and high starch component, the component providing minerals and vitamins, the high-protein and low moisture component, or the high starch component.

6. The method of any preceding claim wherein the one or more edible ingredients are selected from cereal grain, root vegetables, and legumes or a combination or extract thereof.

7. The method of claim 6 wherein the cereal grain is the dry component.

8. The method of claim 6 or 7 wherein the grain is selected from maize (corn), rice, wheat, barley, sorghum, millet, oats, triticale, rye, and/or fonio.

9. The method of claims 6 to 8, wherein the cereal grain is spent grain from fermentation of alcohol (brewers spent grain (BSG).

10. The method of claims 8 to 9, wherein the cereal grain is oats.

11. The method of claims 6 to 10, wherein the one or more edible ingredients comprise, such as 5% wt to 10% wt, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt cereal grain.

12. The method of claim 11, wherein the one or more edible ingredients comprise 15 to 25% wt, optionally 19 to 21% wt of oats.

13. The method of claim 6, wherein the root vegetable is the high-moisture and high starch component and/or the component providing minerals and vitamins.

14. The method of claim 6 or 13, wherein the root vegetable a modified plant stem vegetable, a root-like stem vegetable, or a true root vegetable.

15. The method of claim 14, wherein the modified plant stem vegetable is a Corm (bulbo-tuber/bulbotuber), a Rhizome or a Tuber.

16. The method of claim 15, wherein the Corm is selected from Amorphophallus konjac (konjac), Colocasia esculenta (taro), Eleocharis dulcis (Chinese water chestnut), Ensete spp. (enset), Nymphaea spp. (waterlily), Pteridium esculentum, Sagittaria spp. (arrowhead or wapatoo), Typha spp., Xanthosoma spp. (malanga, cocoyam, tannia, yautia and other names), and/or Colocasia antiquorum (eddoe or Japanese potato).

17. The method of claim 15, wherein the Rhizome is selected from Curcuma longa (turmeric), Panax ginseng (ginseng), Arthropodium spp. (rengarenga, vanilla lily, and others), Canna spp. (canna), Cordyline fruticosa (ti), Maranta arundinacea (arrowroot), Nelumbo nucifera (lotus root), Typha spp. (cattail or bulrush), and/or Zingiber officinale (ginger, galangal).

18. The method of claim 15, wherein the Tuber is selected from Apios americana (hog potato or groundnut), Cyperus esculentus (tigernut or chufa), Dioscorea spp. (yams, ube), Dioscorea polystachya (Chinese yam or white ñame), Helianthus tuberosus (Jerusalem artichoke or sunchoke), Hemerocallis spp. (daylily), Lathyrus tuberosus (earthnut pea), Oxalis tuberosa (oca or New Zealand yam), Plectranthus edulis and P. esculentus (kembili, dazo, and others), Solanum tuberosum (potato), Stachys affinis (Chinese artichoke or crosne), Tropaeolum tuberosum (mashua or añu), and/or Ullucus tuberosus (ulluku).

19. The method of claim 18, wherein the Tuber is of the genus Solanum.

20. The method of claim 19, wherein the Tuber is of the species Solanum tuberosum (potato).

21. The method of claim 20, wherein the Solanum tuberosum is a red skin potato.

22. The method of claims 14 to 21, wherein the root-like stem vegetable is Zamia integrifolia (Florida arrowroot).

23. The method of claims 14 to 21, wherein the true root vegetable is selected from a taproot or a tuberous root.

24. The method of claim 23, wherein the tuberous root vegetable is selected from Amorphophallus galbra (yellow lily yam), Conopodium majus (pignut or earthnut), Dioscorea polystachya (nagaimo, Chinese yam, Korean yam, mountain yam), Hornstedtia scottiana (native ginger), Ipomoea batatas (sweet potato), Ipomoea costata (desert yam), Manihot esculenta (cassava or yuca or manioc), Mirabilis expansa (mauka or chago), Psoralea esculenta (breadroot, tipsin, or prairie turnip), and/or Smallanthus sonchifolius (yacón).

25. The method of claim 23, wherein the taproot vegetable is selected from Arracacia xanthorrhiza (arracacha), Beta vulgaris (beet and mangelwurzel), Brassica spp. (kohlrabi, rutabaga and turnip), Bunium persicum (black cumin), Burdock (Arctium, family Asteraceae), Carrot (Daucus carota subsp. sativus), Celeriac (Apium graveolens rapaceum), Daikon—the large East Asian white radish (Raphanus sativus var. longipinnatus), Dandelion (Taraxacum) spp., and/or Lepidium meyenii (maca).

26. The method of claim 25, wherein the taproot vegetable is Beta vulgaris, optionally subspecies vulgaris.

27. The method of claim 26, wherein the taproot vegetable is B. vulgaris var. conditiva (red beet).

28. The method of claims 6 and 14 to 27, wherein the one or more edible ingredients comprise 5% wt to 60% wt root vegetable, such as 5% wt to 10% wt, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt, optionally 38 to 42% wt.

29. The method of claim 28, wherein the one or more edible ingredients comprise 10 to 30% wt, optionally 15 to 25% wt, optionally 19 to 21% wt of Solanum tuberosum.

30. The method of claim 28 wherein the one or more edible ingredients comprise optionally 15 to 25% wt, optionally 19 to 21% wt of B. vulgaris var. conditiva.

31. The method of claims 29 to 30, wherein the one or more edible ingredients comprise 10 to 30% wt, optionally 15 to 25% wt, optionally 19 to 21% wt of Solanum tuberosum and 10 to 30% wt, optionally 15 to 25% wt, optionally 19 to 21% wt of B. vulgaris var. conditiva.

32. The method of claim 6, wherein the legume is the high-protein and low moisture component and/or the high starch component providing sustained nutrients for the one or more microorganism and providing softness to the texture of the edible product.

33. The method of claim 6 or 32, wherein the legume is a bean selected from the genus of Phaseolus, Pisum, Vigna, Cajanus, Lens, Cicer, Vicia, Arachis, Glycine, Macrotyloma, Mucuna, Lupinus, Ceratonia, Canavalia, Cyamopsis, Lablab, Psophocarpus, Clitoria, Lathyrus, Trifolium, Medicago, Melilotus, and/or Tamarindus.

34. The method of claim 33 wherein the Phaseolus bean is selected from the species P. vulgaris (Kidney Bean, Pinto Bean, Navy Bean Haricot Bean, Black Beans, Borlotti Beans), P. lunatus (Lima Bean), P. coccineus (Runner Bean, Flat Bean), and/or P. acutifolius (Tepary Bean).

35. The method of claim 33 wherein the Pisum bean is selected from P. sativum peas (Green Peas, White Peas, Yellow Peas, Field Peas, Snow Peas, Snap Peas).

36. The method of claim 33 wherein the Vigna bean is selected from V. radiata (Mung Bean), V. mungo (Urad), V. unguiculata (Cowpea, Yardlong bean, Black-eyed Pea), V. aconitifolia (Moth bean), and/or V. angularis (Adzuki bean).

37. The method of claim 33 wherein the Cajanus bean is selected from C. cajan (Pigeon Pea).

38. The method of claim 33 wherein the Lens bean is selected from L. culinaris (Lentil, Red Lentil, Green Lentil, Puy Lentil).

39. The method of claim 33 wherein the Cicer bean is selected from C. arietinum (Chickpea, Garbanzo Bean).

40. The method of claim 33 wherein the Vicia bean is selected from V. faba (Fava Bean, Broad Bean), V. ervilia (Bitter vetch), and/or V. sativa (common vetch).

41. The method of claim 33 wherein the Arachis bean is selected from A. hypogaea (peanut).

42. The method of claim 33 wherein the Glycine bean is selected from G. max (soybean).

43. The method of claim 33 wherein the Macrotyloma bean is selected from M. uniflorum (Horsegram).

44. The method of claim 33 wherein the Mucuna bean is selected from M. pruriens (velvet bean).

45. The method of claim 33 wherein the Lupinus bean is selected from L. albus (white lupin, sweet lupin), L. mutabilis (Tarwi/Andean Lupin), L. hirsutus and/or L. angustifolius.

46. The method of claim 33 wherein Ceratonia the bean is selected from C. siliqua (Carob bean).

47. The method of claim 33 wherein the Canavalia bean is selected from C. gladiate (Sword bean), and/or C. ensiformis (Jack bean).

48. The method of claim 33 wherein the Cyamopsis bean is selected from C. tetragonoloba (Guar bean).

49. The method of claim 33 wherein the Lablab bean is selected from L. purpureus (Hyacinth Bean, lablab bean).

50. The method of claim 33 wherein the Psophocarpus bean is selected from P. tetranoglobulus (winged bean).

51. The method of claim 33 wherein the Clitoria bean is selected from C. ternatea (butterfly pea).

52. The method of claim 33 wherein the Lathyrus bean is selected from L. sativus (grass pea) and/or L. tuberosus (tuberous pea).

53. The method of claim 33 wherein the Trifolium bean is selected from T. repens (white Clover), and/or T. pratense (red clover).

54. The method of claim 33 wherein the Medicago bean is selected from M. sativa (alfalfa).

55. The method of claim 33 wherein the Melilotus bean is selected from M. officinalis (sweet clover).

56. The method of claim 33 wherein the Tamarindus bean is selected from T. indica (tamarind).

57. The method of claim to 6 or 32 to 56, wherein the one or more edible ingredients comprise 5% wt to 60% wt legumes, such as 10% wt to 15% wt, such as 15% wt to 20% wt, such as 20% wt to 25% wt, such as 25% wt to 30% wt, such as 30% wt to 35% wt, such as 35% wt to 40% wt, such as 45% wt to 50% wt, such as 55% wt to 60% wt, optionally 38% wt to 42% wt.

58. The method of claims 6 or 32 to 57 wherein the one or more edible ingredients comprise a legume which contain more than 25% wt of protein.

59. The method of claims 6 or 32 to 58 wherein the one or more edible ingredients comprise a legume which contain more than 25% wt of starch.

60. The method of claims 6 or 32 to 59 wherein the one or more edible ingredients comprise a least two legumes of which at least one legume contains more than 25% wt of protein and at least one legume contains more than 25% wt, such as more than 30% wt, such as between 35% wt to 45% wt of starch.

61. The method of claim 33 wherein the one or more edible ingredients comprise Pisum beans and Lupinus beans.

62. The method of claim 61 wherein the one or more edible ingredients comprise P. sativum peas and L. albus lupin seeds.

63. The method of claim 62 wherein the one or more edible ingredients comprise yellow split P. sativum peas and sweet L. albus lupin seeds.

64. The method of claims 33 or 61 to 63 wherein the one or more edible ingredients comprise 10% wt to 30% wt, optionally 15% wt to 25% wt, optionally 19% wt to 21% wt of yellow split P. sativum peas.

65. The method of claims 33 or 61 to 64, wherein the one or more edible ingredients comprise optionally 15% wt to 25% wt, optionally 19% wt to 21% wt of sweet L. albus lupin seeds.

66. The method of claims 33 or 61 to 65 wherein the one or more edible ingredients comprise 10% wt to 30% wt, optionally 15% wt to 25% wt, optionally 19% wt to 21% wt of yellow split P. sativum peas and 10% wt to 30% wt, optionally 15% wt to 25% wt, optionally 19% wt to 21% wt of sweet L. albus lupin seeds.

67. The method of any preceding claim wherein the one or more edible ingredients comprise oats, Solanum tuberosum, B. vulgaris var. conditiva, P. sativum peas and L. albus lupin seeds.

68. The method of any preceding claim wherein the one or more edible ingredients comprise 10% wt to 30% wt, optionally 15% wt to 25% wt, optionally 19% wt to 21% wt each of oats, potato, red beet, P. sativum peas and L. albus lupin seeds.

69. The method of any preceding claim wherein the one or more edible ingredients further comprise an aqueous extract of oats (oat milk).

70. The method of any preceding claim wherein the one or more edible ingredients further comprise an aqueous extract of almonds (almond milk).

71. The method of any preceding claim wherein the one or more edible ingredients comprise oats and/or BSG and wherein the oats, or and/or BSG prior to forming the fermentation substrate, are pre-processed by milling into a particle size between 2 and 5 mm, optionally between 3 and 4 mm, optionally to an average particle size of 3.5 mm.

72. The method of any preceding claim wherein the one or more edible ingredients comprise potato and wherein the potato, prior to forming the fermentation substrate, is pre-processed by wholly or partially gelatinizing starch by boiling, microwaving or steaming.

73. The method of claim 71 or 72, wherein the oats and potato, prior to forming the fermentation substrate, is further pre-processed by co-grinding in a ratio of 0.5:1 to 1:0,5 of oats:potato, optionally 1:1, until no visible chunks of potato can be observed.

74. The method of any preceding claim wherein the one or more edible ingredients comprise red beet and wherein the red beet, prior to forming the fermentation substrate, is pre-processed by i) boiling and/or steaming and ii) grinding to an average particle size of between 1,7-2,8 mm.

75. The method of any preceding claim wherein the one or more edible ingredients comprise P. sativum peas, and wherein the P. sativum peas, prior to forming the fermentation substrate, is pre-processed by i) drying and splitting the P. sativum peas, ii) microwaving, boiling or steaming the P. sativum peas and iii) grinding the P. sativum peas to an average particle size of between 1,7-2,8 mm.

76. The method of any preceding claim wherein the one or more edible ingredients comprise L. albus lupin seeds, and wherein the lupin seeds, prior to forming the fermentation substrate, is pre-processed by i) soaking the lupin seeds in water to wholly or partially remove toxic or bitter tasting alkaloids, ii) microwaving, boiling or steaming the lupin and iii) grinding the lupin to an average particle size of between 1,7-2,8 mm.

77. The method of any preceding claim further comprising grinding and mixing the pre-processed one or more edible ingredients into a fermentation substrate and optionally pasteurize the fermentation substrate.

78. The method of any preceding claim wherein the one or more edible ingredients prior to forming the fermentation substrate is ground to an average particle size of between 150 μm to 350 μm, optionally 200 μm to 300 μm, optionally 225 μm to 275 μm, optionally around 250 μm.

79. The method of claims 77 to 78, further comprising adjusting the water content to between 35 to 40% wt, optionally 36 to 38% wt in the fermentation substrate.

80. The method of any preceding claim, wherein the one or more microorganisms comprise a GRAS strain.

81. The method of any preceding claim, wherein the one of more microorganisms a fungus.

82. The method of claim 81 wherein the fungus is a filamentous fungus.

83. The method of claim 82 wherein the fungus is a filamentous fungus is of the genus Aspergillus, optionally of the species Aspergillus oryzae and/or Aspergillus sojae.

84. The method of claim 82 wherein the fungus is a filamentous fungus is of the genus Rhizopus optionally of the species Rhizopus oligosporus.

85. The method of any preceding claim, further comprising mixing the one or more microorganisms into the fermentation substrate and allowing the one or more microorganism to colonize and ferment the fermentation substrate at a predetermined time interval, temperature and humidity.

86. The method of claim 85, wherein the fermentation time is between 20 to 90 hours, optionally 30 to 60 hours, optionally 40 to 50 hours.

87. The method of claim 85 or 86, wherein the temperature during incubation is maintained between 20° C. and 40° C., optionally between 25° C. and 35° C., optionally between 29° C. and 31° C.

88. The method of any preceding claim, further comprising rehydrating the fermented fermentation substrate, optionally to a moisture content of between 40% wt to 90% wt, optionally between 50% wt to 80% wt, optionally between 60% wt to 70% wt.

89. The method of any preceding claim, wherein the enriched or improved flavour property is sourness, sweetness, bitterness, saltiness, umami, or a combination thereof.

90. The method of any preceding claim, wherein the enriched or improved texture property is firmness, softness, cohesiveness, juiciness, chewiness, sandiness, or a combination thereof.

91. The method of any preceding claim, wherein the enriched or improved cooking property is improved browning of the fermented edible product (Maillard reaction) when roasting or frying the fermented edible product compared to roasting or frying the edible product prior to fermentation.

92. The method of any preceding claim, wherein the enriched or improved cooking property is improved structure integrity of the fermented edible product compared to the edible product prior to fermentation, where the microorganism biomass binds together the edible ingredients.

93. The method of any preceding claim, wherein the enriched or improved visual property is colour.

94. The method of any preceding claim, wherein the enriched or improved nutritional and/or dietary property is content and/or distribution of protein, lipids, carbohydrates, fibre, vitamins, minerals, amino acids or a combination thereof.

95. A fermented edible product comprising one or more filamentous fungi or the mycelium thereof and 10% wt to 30% wt, optionally 15% wt to 25% wt, optionally 19% wt to 21% wt each of oats, potato, red beet, P. sativum peas and L. albus lupin seeds.

96. The fermented edible product of claim 95 further comprising more than 10% wt of dietary fibres.
